SEQUENCE LISTING (9 sequences)

SEQ ID NO: 1 - DNA, 30 bp, Artificial Sequence
  Forward PCR primer cg10705422
aggaaatgatttatggatttttgttattag

SEQ ID NO: 2 - DNA, 30 bp, Artificial Sequence
  Reverse PCR primer cg10705422
taaccctactacatactcataaaatacaat

SEQ ID NO: 3 - DNA, 25 bp, Artificial Sequence
  PCR primer cg10705422
attaggttagagtatgtaatgaaaa

SEQ ID NO: 4 - DNA, 25 bp, Artificial Sequence
  Forward PCR primer cg17707274
ttgatttggtgttttttgttagtga

SEQ ID NO: 5 - DNA, 26 bp, Artificial Sequence
  Reverse PCR primer cg17707274
ctcaaataaatcacctatttccacat

SEQ ID NO: 6 - DNA, 24 bp, Artificial Sequence
  PCR primer cg17707274
agtgattgtagaaatttatatagt

SEQ ID NO: 7 - DNA, 24 bp, Artificial Sequence
  Forward PCR primer cg26849382
ggagttggaagaggagagttatta

SEQ ID NO: 8 - DNA, 25 bp, Artificial Sequence
  Reverse PCR primer cg26849382
ccttaacactccaaccattattctc

SEQ ID NO: 9 - DNA, 25 bp, Artificial Sequence
  PCR primer cg26849382
aaagtaatatgtaaatttttgtgat

(19) United States
(12) Patent Application Publication
(10) Pub. No.: US 2022/0213556 A1
(43) Pub. Date: Jul. 7, 2022

(54) METHOD AND COMPOSITION FOR DETECTING THYROID CANCER-SPECIFIC DNA METHYLATION BIOMARKER FOR DIAGNOSIS OF THYROID CANCER

(71) Applicants: THE CATHOLIC UNIVERSITY OF KOREA INDUSTRY-ACADEMIC COOPERATION FOUNDATION, Seoul (KR); KOREA RESEARCH INSTITUTE OF BIOSCIENCE AND BIOTECHNOLOGY, Daejeon (KR)

(72) Inventors: Chan Kwon JUNG, Seoul (KR); Yong Sung KIM, Daejeon (KR); Jong Lyul PARK, Daejeon (KR)

(73) Assignees: THE CATHOLIC UNIVERSITY OF KOREA INDUSTRY-ACADEMIC COOPERATION FOUNDATION, Seoul (KR); KOREA RESEARCH INSTITUTE OF BIOSCIENCE AND BIOTECHNOLOGY, Daejeon (KR)

(21) Appl. No.: 17/609,871

(22) PCT Filed: Mar. 26, 2020
(86) PCT No.: PCT/KR2020/004093

§ 371 Date: Nov. 9, 2021

(30) Foreign Application Priority Data
Mar. 29, 2019 (KR) 10-2019-0037358

(51) Int. Cl. C12Q 1/6886; C12Q 1/6858

(52) U.S. Cl. CPC C12Q 1/6886; C12Q 1/6858; C12Q 2600/118; C12Q 2600/112; C12Q 2600/154

(57) ABSTRACT

A method for analyzing the methylation level of a specific CpG site in genomic DNA and a composition suitable to use in the method are provided. The method and composition can provide information useful for diagnosing thyroid cancer or determining the prognosis of patients with thyroid cancer. By using the method and the composition, thyroid cancer can be easily and accurately diagnosed from biological samples at a low cost.

### TECHNICAL FIELD

The present disclosure relates to a method for detecting a thyroid cancer-specific DNA methylation biomarker and a composition thereof in order to provide information necessary for thyroid cancer diagnosis.

### BACKGROUND ART

Papillary thyroid carcinoma (PTC) is the most common thyroid cancer, and its incidence has rapidly increased over the last three decades. There are more than 10 histological variants of PTC that are characterized by disparate molecular and clinical features. The follicular variant is the second most common papillary carcinoma type and consists of an infiltrative and encapsulated form. According to the 2017 World Health Organization (WHO) classification of thyroid tumors, most cases of noninvasive encapsulated follicular variant of papillary carcinoma have been reclassified as noninvasive follicular thyroid neoplasm with papillary-like nuclear features (NIFTP). NIFTP, previously classified as a type of papillary carcinoma, is no longer malignant and has been down-classified as a borderline tumor with uncertain malignant potential. The histologic variants of PTC associated with more aggressive outcomes are tall cell variant, columnar cell variant, and hobnail variant. They have a higher risk of recurrence and disease mortality compared to other variants.

Encapsulated thyroid tumors with histological characteristics of follicle type include follicular adenoma, NIFTP, follicular thyroid carcinoma, and invasive encapsulated follicular variant of PTC. Unlike follicular adenoma and follicular carcinoma, NIFTP and invasive encapsulated follicular variant of PTC have a characteristic nuclear features of PTC. However, these tumors are diagnosed through histological examination after surgery, and they are not differentiated from each other by fine needle aspiration cytology or core needle biopsy performed before surgery, so they are diagnosed as follicular neoplasms. In addition, follicular patterned thyroid tumors often show RAS mutations. Therefore, since it is almost impossible to diagnose these thyroid tumors before surgery accurately, most of these patients undergo surgery for diagnosis and treatment.

DNA methylation is the most well-known epigenetic modification, occurring from the addition of a methyl group to the 5′-position of a cytosine of cytosine-guanine dinucleotides CpG). Inactivation of a tumor suppressor gene may occur via hypermethylation at a promoter region of genes, and oncogenes may be activated by promoter hypomethylation. In addition, recent research studies have revealed that DNA methylation at enhancer or super enhancer regions plays a very important roles in cancer progression via regulation of target gene expression. Most previous studies and The Cancer Genome Atlas (TCGA) project used the Infinium HumanMethylation450 BeadChip methylation Microarray platform (450K, Illumina, San Diego, Calif.) due to its low cost, a small amount of input DNA, simple workflow, and fast sample processing time. However, the Infinium 450K microarray focuses on the coding RNAs loci and lacks coverage of the enhancer regions. In this regard, the previous studies were limited by the low genome coverage of the method. On the other hand, Infinium MethylationEPIC BeadChip (Illumina) is a recently developed platform that covers over 850,000 CpG methylation sites, but research results using it in the thyroid tumor area have not been reported. Therefore, the present inventors were able to develop a new biomarker using the Infinium MethylationEPIC BeadChip technique.

The patents and references mentioned in this specification hereby incorporated by reference to the same extent as if each publication were individually and explicitly specified by reference.

### DISCLOSURE

### Technical Problem

The present inventors have made research efforts to develop molecular diagnostic biomarkers that can be used for positive and malignant diagnosis of thyroid cancer in a low cost and convenient manner. As a result, the present disclosure was completed by discovering methylated biomarkers at CpG sites located in specific genes and experimentally proving that this CpG site methylated biomarker can be used to diagnose thyroid tumors as benign or malignant, as well as predicting metastasis and postoperative recurrence rates.

Accordingly, an objective of the present disclosure is to provide a method of analyzing the methylation level of the thyroid cancer biomarker CpG site of genomic DNA obtained from an analysis target sample in order to provide information necessary for diagnosing thyroid cancer or determining the prognosis of thyroid cancer.

Another objective of the present disclosure is to provide a composition for diagnosing thyroid cancer or determining the prognosis of thyroid cancer, including a substance capable of analyzing the methylation level of the thyroid cancer biomarker CpG site.

The other objectives and technical features of the present disclosure are more specifically set forth by the following detailed description of the disclosure, claims, and drawings.

### Technical Solution

According to one aspect of the present disclosure, in order to provide information necessary for diagnosing thyroid cancer or determining the prognosis of thyroid cancer, the present disclosure provides a method for analyzing the methylation level of a thyroid cancer biomarker CpG site of a genomic DNA obtained from an analysis target sample, in which the thyroid cancer biomarker CpG site is located in a gene selected from the group consisting of: (i) MICAL2 (Microtubule associated monooxygenase, calponin and LIM domain containing 2); (ii) LURAP1L-AS1 (LURAP1L antisense RNA 1); (iii) PKM2 (Pyruvate kinase M2); (iv) LTBP1 (Latent-transforming growth factor beta-binding protein 1); (v) MMP7 (Matrix metalloproteinase-7); (vi) Eukaryotic translation initiation factor 4E (EIF4E); (vii) DIAPH1 (Protein diaphanous homolog 1); and (viii) LOC100507487 (long intergenic non-protein coding RNA 2615.

As used herein, the term “analysis of methylation level” refers to an analysis that measures the methylation status of CpG dinucleotides at a specific region in genomic DNA to be analyzed.

As used herein, the term “methylation state” refers to the presence or absence of 5-methylcytosine in one or more CpG dinucleotides at a specific region in the genomic DNA to be analyzed.

As used herein, the term “hypermethylation” refers to a methylation state in which the amount of 5-methylcytosine in one or more CpG dinucleotides tested in the DNA sequence of the DNA sample is increased compared to the amount of 5-methylcytosine found in the corresponding CpG dinucleotide in the normal control DNA sample.

As used herein, the term “hypomethylation” refers to a methylation state in which the amount of 5-methylcytosine in one or more CpG dinucleotides in the DNA sequence of the DNA sample being tested is reduced compared to the amount of 5-methylcytosine found in the corresponding CpG dinucleotide in the normal control DNA sample.

As used herein, the term “analyzed target” refers to a mammal suspected of having thyroid cancer, preferably means humans.

The present disclosure is based on the discovery that the hypermethylation/hypomethylation status of CpG dinucleotides at specific regions found in specific genes is strongly associated with diagnosis, classification, and prognosis of thyroid cancer

The measurement of methylation status in the present disclosure is performed for one or more CpG dinucleotides in the DNA of a specific region of the specific gene.

According to one embodiment of the present disclosure, the thyroid cancer to biomarker CpG site is one or more CpG sites selected from the group consisting of cg10705422, cg15441605, cg24327132, cg16336556, cg17707274, cg00567113, cg06034194, cg21341586, cg26849382, and cg05763918 represented by Illumina ID in HumanEPIC BeadChip.

In the present disclosure, the thyroid cancer biomarker CpG site may be used in combination with at least two CpG sites selected from the group consisting of cg10705422, cg15441605, cg24327132, cg16336556, cg17707274, cg00567113, cg06034194, cg21341586, cg26849382, and cg05763918 represented by Illumina ID in HumanEPIC BeadChip. For example, a combination of the cg10705422 CpG site and at least one other CpG site; a combination of the cg17707274 CpG site and at least one other CpG site; or a combination of the cg26849382 CpG site and at least one other CpG site may be used. In addition, a combination of the cg10705422 CpG site and the cg17707274 CpG site; a combination of the cg10705422 CpG site and the cg26849382 CpG site; or a combination of the cg17707274 CpG site and the cg26849382 CpG site may be used. Also, a combination of the cg10705422 CpG site, the cg17707274 CpG site, and the cg26849382 CpG site may be used.

According to one embodiment of the present disclosure, when the methylation level of the thyroid cancer biomarker CpG site is hypomethylated, hypomethylated state may be determined to indicate thyroid cancer or a poor prognosis of thyroid cancer.

As used herein, the term “diagnosis” refers to confirming the presence or characteristics of a pathological condition, and for the purposes of the present disclosure, the diagnosis is to determine whether thyroid cancer occurs.

According to one embodiment of the present disclosure, the diagnosis of thyroid cancer may include determining the stage of thyroid cancer.

According to one embodiment of the present disclosure, the prognosis of thyroid cancer may include a recurrence rate of thyroid cancer.

According to another embodiment of the present disclosure, when the methylation level of the thyroid cancer biomarker CpG site is hypomethylated, hypomethylated state may indicate that the recurrence rate of thyroid cancer may be high.

In the present disclosure, the sample is preferably a biological sample, for example, tissue, whole blood, serum, plasma, bodily fluid, urine, cells, cell lysate, or cell culture supernatant but is not limited thereto.

The determination of the methylation status of one or more CpG dinucleotides on DNA may be performed using various known DNA methylation assay methods. For example, there is a method of detecting southern blot or performing PCR amplification after cutting DNA with methylation-sensitive restriction enzyme. A method based on bisulfite treatment includes methylation-specific PCR (MSP) methods and sequencing DNA treated with bisulfite. For example, a simplified genomic sequencing method has been developed to analyze DNA methylation patterns and 5-methylcytosine using bisulfite treatment (Frommer et al., Proc. Natl. Acad. Sci. USA 89: 1827-1831, 1992). In addition, a method for detection by cutting the PCR product amplified from the bisulfite-treated DNA with a restriction enzyme (Sadri & Hornsby, Nucl. Acids Res. 24:5058-5059, 1996), or COBRA (Combined Bisulfite Restriction Analysis) (Xiong & Laird, Nucleic Acids Res. 25:2532-2534, 1997) are known.

According to one embodiment of the present disclosure, the methylation level analysis may include treating the genomic DNA obtained from the sample to be analyzed with bisulfite.

According to one embodiment of the present disclosure, the methylation level analysis may include amplifying the fragment, including the thyroid cancer biomarker CpG site.

Methylation analysis using sequencing of bisulfite-treated DNA is based on the following principle. When methylation occurs at the CpG dinucleotide site, 5-methylcytosine is formed, and this modified base is changed to uracil upon treatment with bisulfite. When treating bisulfite on DNA extracted from the sample, CpG dinucleotide is converted to uracil if methylated and is preserved as cytosine if not methylated. Sequencing of the bisulfite-treated DNA may be preferably performed using a pyrosequencing method. Detailed descriptions of pyrosequencing are known in the prior literature [Ronaghi et al. Science 1998 Jul 17, 281(5375), 363-365; Ronaghi et al. Analytical Biochemistry 1996 Nov 1, 242(1), 84-9; Ronaghi et al. Analytical Biochemistry 2000 Nov 15, 286 (2): 282-288; Nyr, P. Methods Mol Biology 2007, 373, 114].

In addition, the MSP method is a method of designing and using a primer to perform PCR after bisulfite treatment on sample DNA as a different type of primer depending on whether CpG dinucleotide is methylated or not. If the primer binding site is methylated, PCR proceeds with the methylated primer, and if not methylated, PCR proceeds with the normal primer. That is, after treating the sample DNA with an acid salt, PCR is performed using two types of primers at the same time, and the results are compared.

As another method, there is a method of measuring whether methylation is performed using a methylation-sensitive restriction enzyme. A methylation-sensitive restriction enzyme uses a CpG dinucleotide as a site of action, and when this site is methylated, the methylation-sensitive restriction enzyme cannot act as an enzyme. Therefore, when sample DNA is treated with methylation-sensitive restriction enzymes and then amplified with PCR to include enzyme target regions, the restriction enzyme is not applied in the case of methylation so that the DNA is amplified with PCR. However, since the normal region that is not methylated is cut by the restriction enzyme, the DNA is not amplified with PCR, thereby measuring whether a specific DNA site is methylated or not.

Determination of methylation status is performed, for example, in “MethyLight” (a fluorescence-based real-time PCR technique) (Eads et al., Cancer Res. 59:2302-2306, 1999), Ms-SNuPE (Methylation-sensitive Single Nucleotide Primer Extension), Reaction (Gonzalgo & Jones, Nucleic Acids Res. 25:2529-2531, 1997), MSP(methylation-specific PCR) (Herman et al., Proc. Nal Acad. Sci. USA 93:9821-9826, 1996; U.S. Pat. No. 5,786,146), and methylated CpG island amplification (“MCA”; Toyota et al., Cancer Res. 59:2307-12, 1999) can be used alone or in combination with other methods.

According to another aspect of the present disclosure, the present disclosure provides a composition for diagnosing thyroid cancer or determining the prognosis of thyroid cancer, which includes a substance capable of analyzing the methylation level of the thyroid cancer biomarker CpG site located in a gene selected from the group consisting of: (i) MICAL2 (Microtubule associated monooxygenase, calponin and LIM domain containing 2); (ii) LURAP1L-AS1 (LURAP1L antisense RNA 1); (iii) PKM2 (Pyruvate kinase M2); (iv) LTBP1 (Latent-transforming growth factor beta-binding protein 1); (v) Matrix metalloproteinase-7 (MMP7); (vi) Eukaryotic translation initiation factor 4E (EIF4E); (vii) DIAPH1 (Protein diaphanous homolog 1); and (viii) LOC100507487 (long intergenic non-protein coding RNA 2615).

According to one embodiment of the present disclosure, the thyroid cancer biomarker CpG site is one or more CpG sites selected from the group consisting of: cg10705422, cg15441605, cg24327132, cg16336556, cg17707274, cg00567113, cg06034194, cg21341586, cg26849382, and cg05763918 represented by Illumina ID in HumanEPIC BeadChip.

In the present invention, the thyroid cancer biomarker CpG site may be used in combination at least two CpG sites selected from the group consisting of: cg10705422, cg15441605, cg24327132, cg16336556, cg17707274, cg00567113, cg06034194, cg21341586, cg26849382, and cg05763918 represented by Illumina ID in HumanEPIC BeadChip. For example, a combination of the cg10705422 CpG site with one or more other CpG sites; a combination of a cg17707274 CpG site with one or more other CpG sites; or a combination of the cg26849382 CpG site with one or more other CpG sites may be used. Also, a combination of a cg10705422 CpG site with a cg17707274 CpG site; a combination of a cg10705422 CpG site with a cg26849382 CpG site; or a combination of the cg17707274 CpG site with the cg26849382 CpG site may be used. Also, combinations with the cg10705422 CpG site, the cg17707274 CpG site, and the cg26849382 CpG site may be used.

According to one embodiment of the present disclosure, the substance capable of analyzing the methylation level of the thyroid cancer biomarker CpG site may be a primer pair capable of amplifying a fragment, including the CpG site.

According to one embodiment of the present disclosure, the composition may further include a sequencing primer for sequencing the amplification product amplified by the primer pair.

According to one embodiment of the present disclosure, the composition may be provided in the form of a “kit”.

As used herein, the term “kit” refers to a collection of reagents for performing nucleic acid amplification or methylation level analysis.

As used herein, the term “primer” refers to a nucleic acid sequence having a short free 3′-terminal OH group that can form a base pair with complementary templates and serves as a starting point for mold strand radiation. In the present disclosure, the primer has 12 or more consecutive nucleotides hybridized to bisulfite-transformed sequences or complementary sequences thereof.

The primers of the present disclosure may be chemically synthesized using the phosphoramidite solid support method or other well-known methods. Such nucleic acid sequences may also be modified using a number of manners known in the related art. Non-limiting examples of such modifications include methylation, encapsulation, substitution of one or more homologues of natural nucleotides, and modifications between nucleotides, such as uncharged linkages (e.g., methyl phosphonate, phosphotriester, phosphoramidate, carbamate, etc.) or charged linkages (e.g., phosphorothioate, phosphorodithioate, etc.). Nucleic acids may contain one or more additional covalently linked residues, such as proteins (e.g., nucleases, toxins, antibodies, signal peptides, poly-L-lysine, etc.), intercalating agents (e.g., acridine, psoralen, etc.), chelating agents (e.g., metals, radioactive metals, iron, oxidizing metals, etc.), and alkylating agents. In the present disclosure, the primer may be modified using a label capable of directly or indirectly providing a detectable signal. Examples of labels include radioactive isotopes, fluorescent molecules, biotin, and the like.

As used herein, the term “hybridization” refers to the formation of a complex between nucleotide sequences sufficiently complementary to form a complex by Watson Crick base pairing. When a primer “hybridizes” with a template, such a complex (or hybrid) is stable enough, for example, to help the priming function required by DNA polymerase initiating DNA synthesis.

The kit of the present disclosure may further include a reagent capable of performing DNA methylation analysis in addition to the primers or probes, for example, restriction enzymes, DNA polymerases, dNTPs, luciferase, or apyrase.

### Advantageous Effect

The features and advantages of the present disclosure are summarized as follows:

(i) The present disclosure relates to a method for analyzing the methylation level at a specific CpG site in genomic DNA in order to provide information necessary for diagnosing thyroid cancer or determining the prognosis of thyroid cancer and a composition for diagnosing thyroid cancer or determining the prognosis of thyroid cancer.

(ii) According to the present disclosure, thyroid cancer can be easily and accurately diagnosed from a biological sample at a low cost.

### DESCRIPTION OF DRAWINGS

FIG. 1a relates to thyroid tumor-specifically differentiated and methylated CpGs (DMCs, differentially methylated CpGs) as measured by EPIC BeadChip and Infinium HumanMethylation 450K bead array.

FIG. 1b is a result of autonomous hierarchical clustering analysis of 34 normal thyroid tissue and tumor tissue samples using CpG (DMC) sites that are differentiated and methylated in a thyroid tumor-specific manner. Thyroid cancer-specific DMCs were selected based on p-values (<0.005) and methylation differences (>0.2 or <0.2) between normal thyroid tissue and thyroid tumor tissue samples. Columns represent each case, and each line represents CpG sites. Red and blue indicate high and low methylation levels, respectively. PTC: papillary thyroid carcinoma; EFV: encapsulated follicular variant; NIFTP: Non-invasive follicular thyroid neoplasm with papillary-like nuclear features.

FIG. 2a is a result of measuring the correlation between DNA methylation level and gene expression level in normal thyroid tissue and thyroid tumor tissue obtained from the TCGA dataset. The mRNA expression levels of hypermethylated genes are significantly up-regulated in the tumor column compared to normal tissues. The mRNA expression of hypomethylated genes is significantly up-regulated in tumor tissues compared to normal tissues.

FIG. 2b shows the distribution characteristics of hypomethylated and hypermethylated differentially methylated CpG (DMCs) and the results of known motif analysis. Panel A is the CpG relationship. Panel B shows the genomic distribution of DMC loci. Panel C shows the DNase sensitivity of the DMC loci. Regions within 100 bp or −100 bp flanking sites of hypomethylated or hypermethylated CpG sites, respectively, were used for known motif analysis. The 10 most important motifs for hypermethylated DMC in panel D or hypomethylated DMC in panel E were compared with known TF-binding sites. Shore, ˜0-2 kb from CGI; Shelf, ˜2-4 kb from CGI; Open Sea, >4 kb from CGI.

FIG. 3a is a heat map for NIFTP-specific hypomethylated CpG sites and hypermethylated CpG sites. NIFTP-specific DMCs were selected based on p-value and methylation differences. Columns represent cases, and lines represent CpG sites. Red and blue indicate high and low methylation levels, respectively.

FIG. 3b shows the results of quantitative analysis of DNA methylation by the pyrosequencing. Representative pyrograms show the methylation levels of cg10705422 (A), cg17707274 (B), and cg26849382 (C).

FIG. 4 shows the evaluation results of candidate methylation markers by pyrosequencing in a new independent study population. Methylation levels of cg10705422 (A), cg17707274 (B), and cg26849382 (C) were quantified by pyrosequencing using a total of 293 formalin-fixed paraffin-embedded tissue samples. FA (follicular adenoma); FTC (follicular thyroid carcinoma); NIFTP (non-invasive follicular thyroid neoplasm with papillary-like nuclear features); HA (Hurthle cell adenoma); HCC (Hurthle cell carcinoma); PTC (papillary thyroid carcinoma); IEFV (invasive encapsulated follicular variant); TCV (tall cell variant).

FIG. 5 shows the results of performing a receiver operating characteristic curve (ROC) analysis on three selected DNA methylation markers to distinguish non-malignant tumors that are not malignant [(follicular adenoma), (Hurthle cell adenoma), and (non-invasive follicular thyroid neoplasm with papillary-like nuclear features)] from malignant tumors [(thyroid papillary carcinoma). (papillary thyroid carcinoma), and (Hurthle cell carcinoma)]. The area under the curve (AUC) indicates the probability that a classifier ranks a randomly selected positive example higher than a randomly selected negative example. The gray curve is the 95% confidence boundary. Criteria for low DNA methylation levels of cg10705422 (A), cg17707274 (B), and cg26849382 (C) were established using AUC.

FIG. 6 shows the results of measuring the diagnostic performance in the case of combining three DNA methylation markers, cg10705422, cg17707274, and cg26849382 in a thyroid tumor. Thyroid tumors were divided into four groups, all hypermethylated (group 1), only one hypomethylated (group 2), two hypomethylated (group 3), and all three hypomethylated (group 4) for the three DNA methylation markers, respectively. The four groups were classified according to the distribution and histopathological types of tumors (Panel A), tumor recurrence risk (Panel B), tumor recurrence or persistence (Panel C), and tumor stage (Panel D). In panel A, group 4 had the highest distribution in papillary carcinoma. In panel B, the high-risk group for recurrence after surgical treatment was observed the most in group 4 and the lowest in group 1. In panel C, the actual recurrence and persistence of tumors occurred the most in group 4 and the lowest in group 1. In panel D, stage 4 cancer was observed only in group 4. Given these characteristics, the patient belonging to group 4 can be predicted to have the worst prognosis, and the patient belonging to group 1 will have the best prognosis. FA (follicular adenoma); NIFTP (non-invasive follicular thyroid neoplasm with papillary-like nuclear features); HA (Hurthle cell adenoma); PTC (papillary thyroid carcinoma).

### BEST MODE

The specific examples described herein are meant to represent preferred embodiments or examples of the present disclosure, and the scope of the present disclosure is not limited thereby. It will be apparent to those skilled in the art that modifications and other uses of the present disclosure do not depart from the scope of the disclosure as set forth in the claims herein.

### EXAMPLE

### Experimental Method

### 1. Subject Study

For profiling of DNA methylation in the present disclosure, 34 frozen tissue samples containing matched normal (n=7), NIFTP (n=6), invasive EFVPTC (n=3), classic PTC (n=11), large cell variant (TCV, tall cell variant), and PTC (n=7) were used. In addition, formalin-fixed and paraffin-embedded tissue samples were used to validate the selected DNA markers by pyrosequencing. Pathological slides from all samples were reviewed and classified according to the 2017 World Health Organization classification of tumors of endocrine organs (Lloyd R V, Osamura R Y, Kloppel G, Rosai J 2017 WHO Classification of Tumors of Endocrine Organs, Vol 10. fourth ed. WHO Press, Geneva, Switzerland). The validation population was follicular adenoma (n=61), Hurthle cell adenoma (n=24), NIFTP (n=56), papillary carcinoma (n=120), follicular carcinoma (n=27) and Hurthle cell carcinoma (n=5). Tumors stages were categorized according to the 8th edition of the American Joint Committee on Cancer (AJCC) staging manual. The risk of recurrence was evaluated based on the American Thyroid Association (ATA) classification for risk of recurrence. The exact number of patients and the basic characteristics of the patients are presented in Table 1 below.

TABLE 1

| | Fresh frozen samples for | FFPE samples for |
|---|---|---|
| Characteristic | discovery | validation |
| Sample | 34 | 293 |
| Year of collection | 2013-2016 | 2008-2017 |
| Age years at diagnosis, mean (range) | 43.7 (26-70) | 47.5 (19-83) |
| Sex | | |
| Female | 17 | 182 |
| Male | 9 | 111 |
| Tumor size (cm), mean (range) | 2.1 (1.1-5.0) | 2.4 (1.0-9.0) |
| Pathologic diagnosis | | |
| Matched normal | 7 | 0 |
| Follicular adenoma | 0 | 61 |
| Hürthle cell adenoma | 0 | 24 |
| NIFTP | 6 | 56 |
| PTC, invasive encapsulated follicular variant | 3 | 23 |
| PTC, classic type | 11 | 48 |
| PTC, tall cell variant | 7 | 45 |
| PTC, columnar cell variant | 0 | 2 |
| PTC, hobnail variant | 0 | 1 |
| PTC, diffuse sclerosing variant | 0 | 1 |
| Follicular carcinoma, minimally invasive | 0 | 14 |
| Follicular carcinoma, encapsulated angioinvasive | 0 | 12 |
| Follicular carcinoma, widely inivasive | 0 | 1 |
| Hürthle cell carcinoma, minimally invasive | 0 | 4 |
| Hürthle cell carcinoma, encapsulated angioinvasive | 0 | 1 |

FFPE, formalin-fixed paraffin-embedded; NIFTP, noninvasive follicular thyroid neoplasm with papillary-like nuclear features; PTC, papillary thyroid carcinoma.

### 2. DNA Isolation and BRAF Mutation Analysis

Genomic DNA was isolated from frozen tissue and 10 μm-thick paraffin-embedded tissue using the RecoverAll™ Total Nucleic Acid Isolation Kit (Life Technologies, Carlsbad, Calif., USA) according to the manufacturer's instructions. Quantitative and qualitative analysis was performed on the extracted genomic DNA using an ND-1000 spectrophotometer (Thermo Fisher Scientific, Waltham, Mass., USA). After amplifying the extracted DNA by PCR, the sequencing analysis of BRAF exon 15 was performed by the previously described direct sequencing method of amplicons (Cho U et al., 2017 Mod Pathol 30:810-825; Jung C K et al., 2018 Hum Pathol 81: 9-17) was used.

### 3. DNA Methylation Microarray Experiments and Data Analysis

Methylation array experiments were performed using an EPIC BeadChip (Illumina) according to the manufacturer's instructions. Briefly, 500 ng of genomic DNA collected from thyroid normal and thyroid tumor tissues was treated with 20 μl of sodium bisulfite solution included in the EZ DNA Methylation-Gold Kit (Zymo Research, Orange, Calif.). Bisulfite-converted DNA (4 μl) was amplified using the Infinium Methylation Assay kit (Illumina). Amplified DNA was hybridized to an EPIC BeadChip and scanned with the Illumina iSCAN system. CpG methylation values were calculated as average-β values using the minfi package of R software (version 1.26.2). A functional normalization method was used to remove technical variations (Fortin J P et al., 2014 Genome Biol 15: 503). Measurements with detection P-values with <0.05 were considered to have a significant signal above the background. All primary methylation array data were deposited in the GEO database under accession number GSE121377.

### 4. Published RNA Sequencing and DNA Methylation Data Collection

Public RNA sequencing data of thyroid normal and PTC samples were obtained from the TCGA data set (https://portal.gdc.cancer.gov/) to estimate the correlation between DNA methylation and gene expression in PTC. Additionally, Infinium HumanMethylation 450K data was downloaded from the TCGA data set, and the top 10 candidate DNA methylation markers selected by the inventor were verified.

### 5. Motif and Gene Ontology Analysis

Analysis of sequencing motif was performed using a HOMER package (version 4.10) with the default parameter settings, using thyroid cancer-specific hypomethylated or hypermethylated sites located at DNase-sensitive regions. Regions for motif analyses were defined as 100 bp upstream to 100 bp downstream of the differently methylated CpGs (DMCs). Gene ontology analysis was performed using DMCs linked with 5′-regulatory regions to predict the function of DMC-linked genes.

In this case, each cell may be allocated respective storage space, and the map feature point information corresponding to the position of the corresponding cell may be stored in the corresponding storage space.

### 6. DNA Methylation Analysis by Pyrosequencing

A pyrosequencing technique was employed to validate the selected DNA methylation markers in an independent cohort. Briefly, 500 ng of total DNA from each of the paraffin-embedded tissue sections was used for bisulfite conversion using the EZ DNA Methylation Gold kit (Zymo Research, Orange, Calif., USA). Each sample was eluted using 20 ul elution buffer from the kit. Next, 1 μl of bisulfite-converted DNA was used in a PCR mixture containing primer sets and 2× Master Mix (Doctor Protein, Seoul, Korea) and amplified using a GeneAmp PCR system 9700 (Applied Biosystems, Waltham, Mass., USA). For pyrosequencing, forward, reverse, and sequencing primers were designed using PSQ Assay Design v2.0.1.15 (Biotage, Kungsgatan, Sweden), and then standard pyrosequencing was performed. Briefly, 20 μl of PCR product was immobilized on 3 μl of Streptavidin Sepharose High Performance (GE Health-care Bio-Sciences, Uppsala, Sweden) and annealed with sequencing primer for 10 minutes at 80° C. Finally, the generated pyrograms were analyzed using PyroMark analysis software (Biotage). Sequences for the primer sets (Bioneer, Daejeon, Korea) are shown in Table 2.

TABLE 2

| | | | Annealing |
|---|---|---|---|
| primer | | | temperature/ |
| designation | direction | order | cycle count |
| cg10705422 | F | 5′-AGGAAATGATTTATGGATTTTTGTTATTAG-3′ | 57° C./40 |
| | | (SEQ ID NO: 1) | |
| | R | 5′-biotin- | |
| | | TAACCCTACTACATACTCATAAAATACAAT-3′ | |
| | | (SEQ ID NO: 2) | |
| | S | 5′-ATTAGGTTAGAGTATGTAATGAAAA-3′ | |
| | | (SEQ ID NO: 3) | |
| cg17707274 | F | 5′-TTGATTTGGTGTTTTTTGTTAGTGA-3′ | 57° C./40 |
| | | (SEQ ID NO: 4) | |
| | R | 5′-biotin-CTCAAATAAATCACCTATTTCCACAT-3' | |
| | | (SEQ ID NO: 5) | |
| | S | 5′-AGTGATTGTAGAAATTTATATAGT-3′ | |
| | | (SEQ ID NO: 6) | |
| cg26849382 | F | 5′-GGAGTTGGAAGAGGAGAGTTATTA-3′ | 60° C./40 |
| | | (SEQ ID NO: 7) | |
| | R | 5′-biotin-CCTTAACACTCCAACCATTATTCTC-3′ | |
| | | (SEQ ID NO: 8) | |
| | S | 5′-AAAGTAATATGTAAATTTTTGTGAT-3′ | |
| | | (SEQ ID NO: 9) | |

### 7. Statistical Analysis

Student's t-test and ANOVA analysis were used to evaluate the significance of differences in gene expression and DNA methylation levels between normal and thyroid cancer tissues or between NIFTP and other subtypes in thyroid tumors. The correlation between clinicopathologic features and methylation levels were analyzed using parametric (chi-squared test) and non-parametric (Fisher's exact) assessments. Through logistic regression analysis, clinicopathological variables and the degree of DNA methylation were compared with clinical results. Using Pearson's correlation method, hierarchical clustering was analyzed with Multiple Experiment Viewer (MEV) software (version 4.8.1). The receiver operating characteristic (ROC) and the respective area under the ROC curve (AUC) were calculated for each DNA methylation marker, using the ROCR package of R software (version 3.4.0).

### Experiment Result

### 1. Identification of Thyroid Tumor-Specific Differentially Methylated CpG Sites

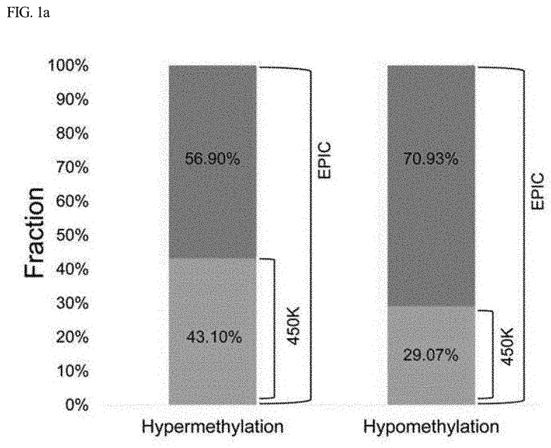

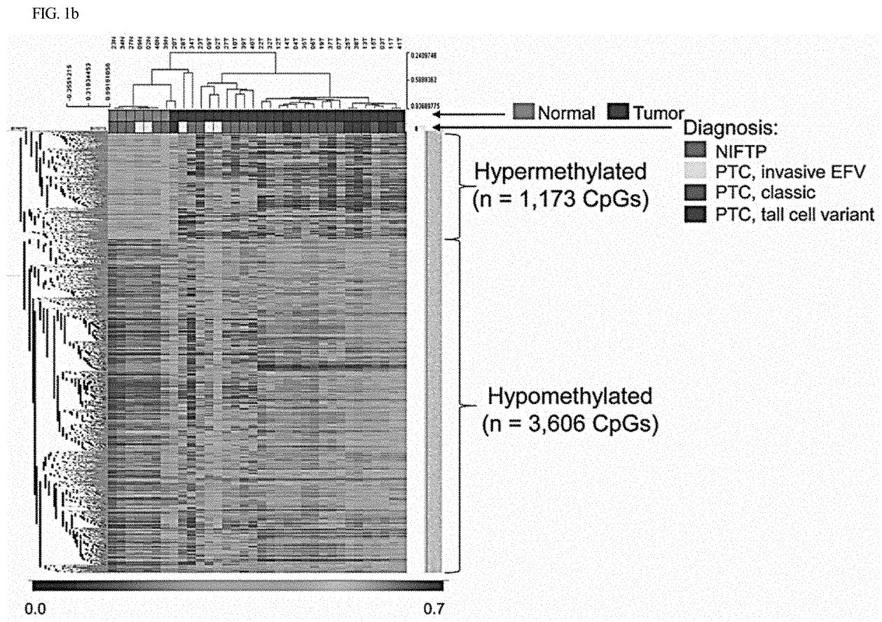

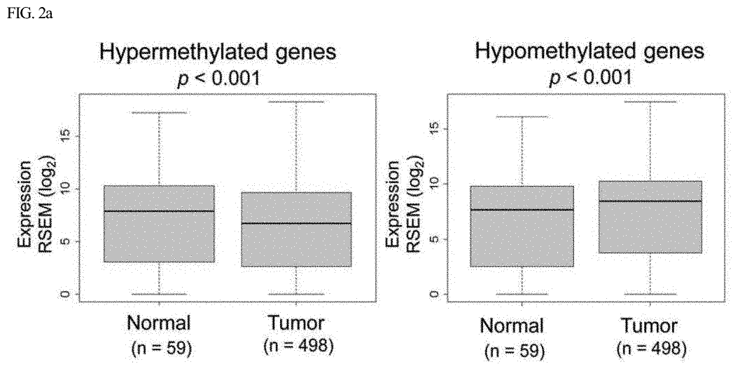

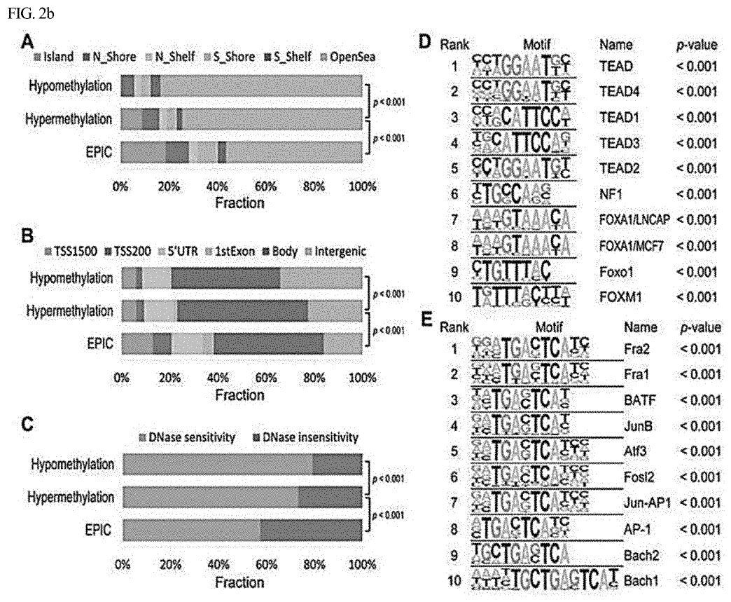

To identify thyroid tumor-specific DMCs, the p-value and methylation differences between thyroid normal (n=7) and thyroid tumor (n=27) samples were calculated. Two criteria were applied: (1) p-value <0.005 and (2) methylation differences >0.2 or <−0.2 (average-β scale) between thyroid normal and thyroid tumor samples. As a result, 3,606 hypomethylated CpG sites and 1,173 hypermethylated CpG sites were selected. Out of the selected DMCs, more than half of the DMCs are novel loci (70.93% hypomethylated DMC and 56.90% hypermethylated DMCs), compared to the Infinium HumanMethylation 450K bead array (FIG. 1a). Unsupervised hierarchical clustering of the DNA methylation of the DMC candidates is shown in FIG. 1b. RNA-sequencing data of thyroid normal and thyroid tumor tissues were collected from the TCGA dataset to estimate the correlation between DNA methylation and gene expression. Out of the DMCs, 318 hypomethylated DMCs and 114 hypermethylated DMCs were located in the 5′-regulatory regions (promoter regions) of 266 and 88 genes, respectively. Next, DNA methylation levels and mRNA expression levels of 88 and 226 genes were evaluated in the thyroid normal and thyroid tumor tissues. As a result, the degree of DNA methylation and the degree of gene expression showed a negative correlation (FIG. 2a).

### 2. Features of Thyroid Cancer-Specific Differentially Methylated CpG Loci

The present disclosure analyzed the distribution features of the thyroid cancer-specific DMCs based on the CpG island relation, gene structure, and DNase sensitivity. CpG island relations were classified into four categories: islands, shore (up to 2 kb from CpG island), shelves (2 to 4 kb from CpG island), and open sea (more than 4 kb from CpG island). Compared to the reference distribution on the EPIC bead array, it was confirmed that hypomethylated DMC or hypermethylated DMC in thyroid cancer were enriched in the open sea area (see panel A of FIG. 2b). Gene structures were classified into the following categories: TSS1500, TSS200, 1stExon, Body, intergenic. As a result of the experiment, the hypomethylated DMCs and the hypermethylated DMCs were predominantly located in the intergenic loci, compared with the EPIC bead array (see panel B of FIG. 2b). When DMCs were separated into two categories based on DNase sensitivity, both hypomethylated DMCs and hypermethylated DMCs were enriched on the DNase-sensitive loci (panel C of FIG. 2b).

Transcription factors (TFs) are proteins with DNA binding activity that are involved in the regulation of transcription. In general, transcription factors modulate gene expression by binding to gene promoter regions or distal regions, called enhancers. The distance between the TFBS and a transcription start site (TSS) of a gene regulated by the TF may be up to several megabases and depends on the chromatin structure of the region. An enrichment analysis of known binding motifs was performed to examine whether thyroid cancer-specific DMCs were associated with TFBS.

Sequences within 100 bp upstream or 100 bp downstream flanking each of the hypomethylated CpG and hypermethylated CpG sites located at the DNase-sensitive loci was used for the enrichment analysis of known binding motifs. As a result, it was revealed that hypermethylated CpG sites were enriched in the TEAD TF family, and hypomethylated DMCs were enriched in Fra2, FraI, BATF, and JunB TF. The top 10 binding motifs selected from hypomethylated CpG sites and hypermethylated CpG sites are shown in panel D of FIG. 2b and panel E of FIG. 2b, respectively.

### 3. Identification of NIFTP-Specific DNA Methylation Markers

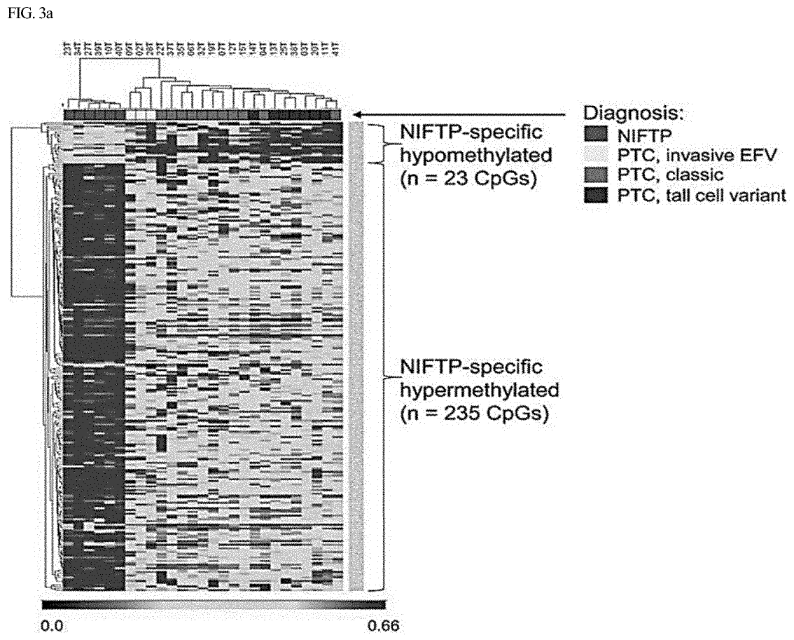

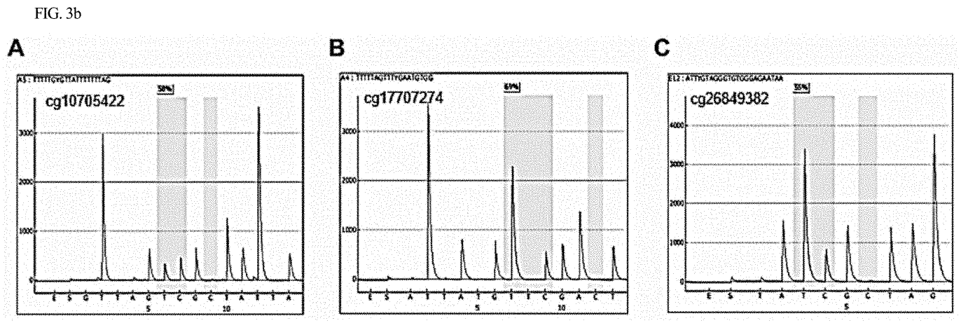

Methylation differences and p-values were calculated to select NIFTP-specific DNA methylation markers. The following two criteria were applied: (1) p-value <0.005 and methylation difference >0.3 or <−0.3 (average-β scale) between NIFTP (n=6) and PTC (n=21), and (2) p-value <0.005 and methylation differences >0.2 or <0.2 (average-β scale) between NIFTP (n=6) and invasive EFVPTC (n=3). As a result, 23 hypomethylated CpG sites and 235 hypermethylated CpG sites were selected, respectively. Unsupervised hierarchical clustering analysis with these DMC candidates was performed (see FIG. 3a). The top 10 DNA methylation candidates were selected in an independent cohort using DNA methylation differences and AUC values to discriminate NIFTP from PTCs (classic PTC, invasive EFVPTC, TCVPTC). The top 10 DNA methylation markers are summarized in Table 3. Since four of the top 10 DNA methylation candidate markers were included in the Infinium HumanMethylation 450K platform, the methylation patterns of candidate DNA methylation markers in the TCGA study cohort could be analyzed. All four DNA methylation markers used for validation showed hypomethylation in papillary carcinoma regardless of histological type.

TABLE 3

| | Delta-b | Delta-b | P-value | P-value | |
|---|---|---|---|---|---|
| | (NIFTP vs | (NIFTP vs | (NIFTP vs | (NIFTP vs | |
| Illumina Id^{a} | other) | IEFVPTC) | other) | IEFVPTC) | AUC |
| cg10705422 | 0.59 | 0.38 | 1.60E−10 | 9.47E−04 | 1 |
| cg15441605 | 0.5 | 0.42 | 1.45E−11 | 4.68E−05 | 1 |
| cg24327132 | 0.59 | 0.34 | 1.23E−10 | 1.94E−03 | 1 |
| cg16336556 | 0.51 | 0.4 | 1.14E−10 | 3.43E−04 | 1 |
| cg17707274 | 0.53 | 0.37 | 1.73E−11 | 1.49E−03 | 1 |
| cg00567113 | 0.48 | 0.42 | 1.30E−11 | 4.17E−03 | 1 |
| cg06034194 | 0.46 | 0.43 | 7.49E−11 | 7.96E−04 | 1 |
| cg21341586 | 0.51 | 0.38 | 2.38E−06 | 3.58E−03 | 1 |
| cg26849382 | 0.52 | 0.37 | 8.00E−08 | 4.81E−03 | 1 |
| cg05763918 | 0.48 | 0.4 | 1.83E−09 | 4.01E−03 | 1 |

| | | | | | Gene |
|---|---|---|---|---|---|
| | | | | CpG | feature |
| Illumina Id^{a} | Chromosome | Mapinfo^{b} | Symbol | island^{c} | group |
| cg10705422 | chr11 | 12188825 | MICAL2 | OpenSea | Body |
| cg15441605 | chr9 | 12814643 | LURAPIL-AS1 | OpenSea | TSS1500 |
| cg24327132 | chr15 | 72520632 | PKM2 | N_Shore | 5′UTR |
| cg16336556 | chr2 | 33295138 | LTBP1 | OpenSea | Body |
| cg17707274 | chr11 | 1.02E+08 | MMP7 | OpenSea | TSS200 |
| cg00567113 | chr3 | 87382813 | | OpenSea | |
| cg06034194 | chr9 | 12814626 | LURAPIL-AS1 | OpenSea | TSS1500 |
| cg21341586 | chr4 | 99851281 | EIF4E | S_Shore | 5′UTR |
| cg26849382 | chr5 | 1.41E+08 | DIAPH1 | OpenSea | Body |
| cg05763918 | chr4 | 1.29E+08 | LOC100507487 | OpenSea | Body |

In Table 3 above, a; Illumina ID is a specific identification number in “HumanEPIC BeadChip.”

b; Mapinfo indicates the genomic location of the human reference genome 37 (GRCh37/hg19), the Genome Reference Consortium on Mar. 3, 2009.

c; Shore and shelf refer to regions flanking to CpG islands (2-kb and 4-kb regions flanking to CpG islands, respectively). N and S mean upstream and downstream of the CpG island, respectively.

NIFTP: non-invasive follicular thyroid neoplasm with papillary-like nuclear features

IEFVPTC, IEFV: Invasive encapsulated follicular variant of papillary thyroid carcinoma

### 4. Validation of Potential DNA Methylation Markers in an Independent Cohort by Pyrosequencing

The NIFTP-specific DNA methylation markers from the microarray data were evaluated with a bisulfite modification-based pyrosequencing assay of 293 paraffin tissue samples included of follicular adenoma (n=61), Hurthle cell adenoma (n=24), NIFTP (n=56), papillary carcinoma (n=120), follicular carcinoma (n=27), and Hurthle cell carcinoma (n=5). Among the top 10 candidate DNA methylation markers, pyrosequencing designs were performed for cg10705422, cg17707274, and cg26849382. Each representative pyrogram is shown in FIG. 3b.

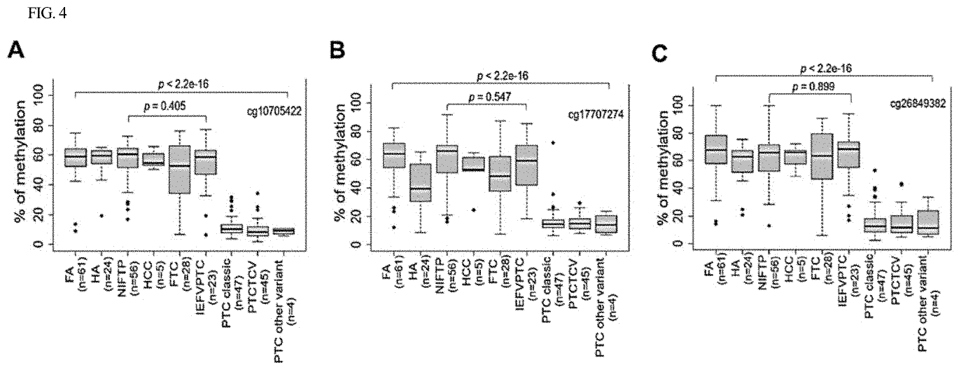

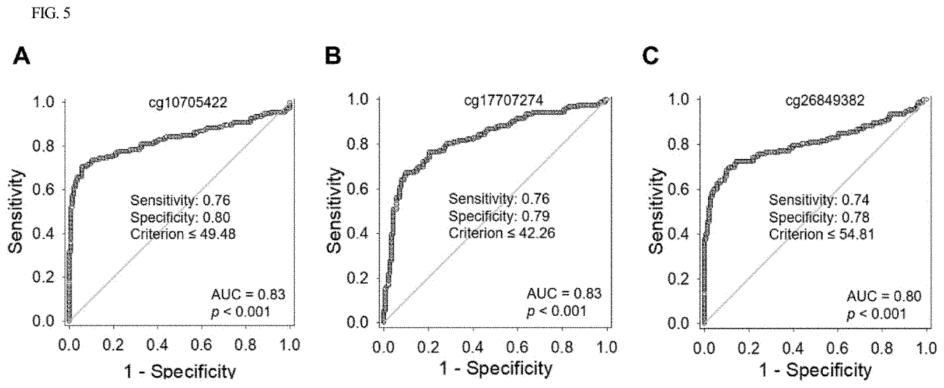

The three candidate DNA methylation markers were hypomethylated at much lower levels in papillary carcinoma than in other thyroid tumors (FIG. 4). The three selected DNA methylation markers were further evaluated for their capacity to differentiate nonmalignant (follicular adenoma, Hurthle cell adenoma, NIFTP) thyroid tumors from malignant (papillary carcinoma, follicular carcinoma, and Hurthle cell carcinoma) thyroid tumors were evaluated. To estimate the AUC value, ROC analysis was performed to confirm good sensitivity and specificity. The AUC values of cg10705422, cg17707274, and cg26849382 were 0.83, 0.83, and 0.80, respectively (see FIG. 5). Optimal cut-off values are described in FIG. 5.

### 5. Clinicopathologic Utility of Three DNA Methylation Markers

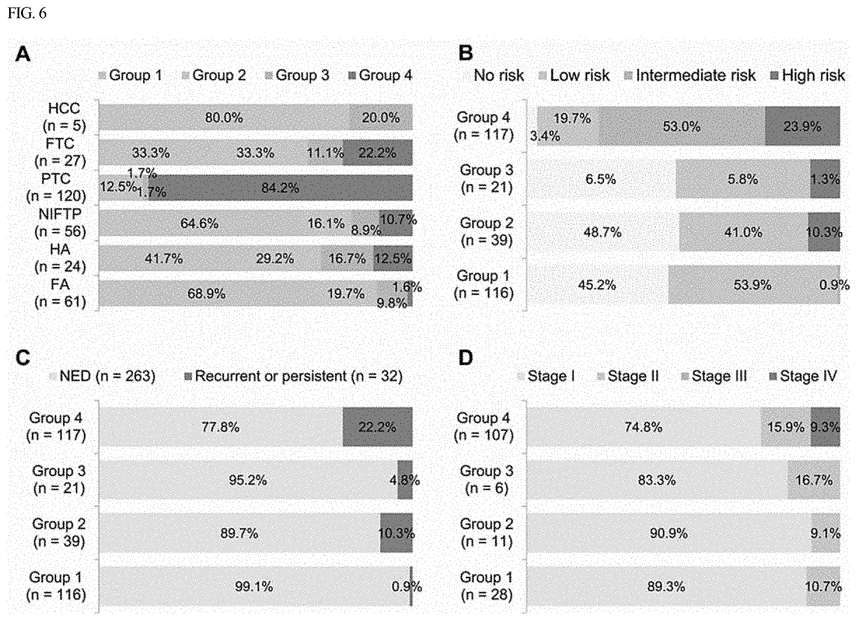

All patients were classified into hypomethylation and hypermethylation according to the different levels of methylation of three DNA methylation markers, and then each of the three DNA methylation markers was combined and classified into 4 groups (FIG. 6): all hypermethylated (group 1), only one hypomethylated (Group 2), two hypomethylated (group 3), all three hypomethylated (group 4). As a result, most of the papillary carcinoma patients belonged to group 4. Most of the patients with recurrent or persistent thyroid cancer belonged to group 4. All stage 4 patients at the time of thyroid cancer surgery belonged to group 4. In multivariate logistic regression analysis, low levels of methylation of the three DNA methylation markers had a significant correlation with tumor recurrence or persistence (odds ratio=3.860; 95%, significance level=1.194-12.475), and also a significant association with remote metastasis. (odds ratio=4.009; 95% significance level=1.098-14.632). Table 4 shows the results of multivariate logistic regression analysis of prognostic factors related to clinical outcomes in 152 differentiated thyroid cancer patients.

TABLE 4

| | Recurrent or persistent | | | | | |
|---|---|---|---|---|---|---|
| | disease^{a} | | | Distant metastasis^{b} | | |
| | Adjusted OR [CI] | | p | Adjusted OR [CI] | | p |
| Age ≥55 years | 2.005 | [0.851-4.725] | 0.112 | 2.975 | [1.193-7.417] | 0.019 |
| Male sex | 0.485 | [0.183-1.289] | 0.147 | 0.315 | [0.106-0.942] | 0.039 |
| PTC | 0.473 | [0.113-1.977] | 0.305 | 0.341 | [0.072-1.608] | 0.174 |
| Aggressive histology^{c} | 3.421 | [1.150-10.180] | 0.027 | 4.624 | [1.307-16.356] | 0.018 |
| Multifocal tumor | 1.278 | [0.505-3.233] | 0.605 | 1.185 | [0.428-3.280] | 0.744 |
| BRAF^{V600E} mutation | 3.421 | [1.460-16.718] | 0.010 | 0.212 | [0.059-0.763] | 0.018 |
| Hypomethylation of three | 3.860 | [1.194-12.475] | 0.024 | 4.009 | [1.098-14.632] | 0.036 |
| DNA methylation markers | | | | | | |

^{a}Includes biochemical (n = 2) and structural locoregional recurrence (n = 3), and distant metastasis (n = 27).
^{b}Includes synchronous (n = 20) and metachronous (n = 7) distant metastasis.
^{c}Includes TCV (n = 45), columnar cell variant (n = 2), and hobnail variant (n = 1) of PTC, encapsulated angioinvasive FTC with extensive angioinvasion (n = 3), and widely invasive FTC (n = 1).
CI, 95% confidence interval; FTC, follicular thyroid carcinoma; OR, odds ratio; TCV, tall cell variant.

Table 5 shows the results of additional analysis for thyroid papillary carcinoma patients only. Low levels of methylation of three methylation markers (cg10705422, cg17707274, cg26849382) had a significant association with histologic variants (P<0.001), extrathyroidal extension (P<0.001), multifocality (P<0.001), and lymph node metastasis (P<0.001), BRAF V600E mutation (P<0.001), tumor recurrence and persistence (P=0.004), and increased risk of recurrence (P<0.001).

TABLE 5

| | cg10705422 | | | | | cg17707274 | | | | | cg26849382 | | | | |
|---|---|---|---|---|---|---|---|---|---|---|---|---|---|---|---|
| | Low | | High | | | Low | | High | | | Low | | High | | |
| Characteristic | methylation | | methylation | | p | methylation | | methylation | | p | methylation | | methylation | | p |
| Age (years) | | | | | 0.319 | | | | | 0.667 | | | | | 0.319 |
| <55 | 73 | (88.0%) | 10 | (12.0%) | | 72 | (86.7%) | 11 | (13.3%) | | 73 | (88.0%) | 10 | (12.0%) | |
| ≥55 | 30 | (81.1%) | 7 | (18.9%) | | 31 | (83.8%) | 6 | (16.2%) | | 30 | (81.1%) | 7 | (81.1%) | |
| Sex | | | | | 0.277 | | | | | 0.969 | | | | | 0.277 |
| Male | 51 | (89.5%) | 6 | (10.5%) | | 49 | (86.0%) | 8 | (14.0%) | | 51 | (89.5%) | 6 | (10.5%) | |
| Female | 52 | (82.5%) | 11 | (17.5%) | | 54 | (85.7%) | 9 | (14.3%) | | 52 | (82.5%) | 11 | (17.5%) | |
| Tumor size (cm) | 1.9 ± 1.1 | | 2.3 ± 1.4 | | 0.203 | 1.9 ± 1.1 | | 2.4 ± 1.4 | | 0.099 | 1.9 ± 1.1 | | 2.3 ± 1.4 | | 0.203 |
| Histologic | | | | | <0.001 | | | | | <0.001 | | | | | <0.001 |
| subtypes | | | | | | | | | | | | | | | |
| Classic | 48 | (100%) | 0 | | | 47 | (97.9%) | 1 | (2.1%) | | 48 | (100%) | 0 | | |
| IEFV | 6 | (26.1%) | 17 | (73.94%) | | 7 | (30.4%) | 16 | (69.6%) | | 6 | (26.1%) | 17 | (73.9%) | |
| TCV | 45 | (100%) | 0 | | | 45 | (100%) | 0 | | | 45 | (100%) | 0 | | |
| Other | 4 | (100%) | 0 | | | 4 | (100%) | 0 | | | | | | | |
| Histologic | | | | | <0.001 | | | | | <0.001 | | | | | <0.001 |
| aggressiveness | | | | | | | | | | | | | | | |
| Nonaggressive | 55 | (76.4%) | 17 | (23.6%) | | 55 | (76.4%) | 17 | (23.6%) | | 55 | (76.4%) | 17 | (23.6%) | |
| variant | | | | | | | | | | | | | | | |
| Aggressive | 48 | (100%) | 0 | | | 48 | (100%) | 0 | | | 48 | (100%) | 0 | | |
| variant | | | | | | | | | | | | | | | |
| Extrathyroidal | | | | | <0.001 | | | | | <0.001 | | | | | <0.001 |
| extension | | | | | | | | | | | | | | | |
| Absent | 24 | (60.0%) | 16 | (40.0%) | | 24 | (60.0%) | 16 | (40.0%) | | 24 | (60.0%) | 16 | (40.0%) | |
| Microscopic | 61 | (98.4%) | 1 | (1.6%) | | 61 | (98.4%) | 1 | (1.6%) | | 61 | (98.4%) | 1 | (1.6%) | |
| Gross | 18 | (100%) | 0 | | | 18 | (100%) | 0 | | | 18 | (100%) | 0 | | |
| Multifocality | | | | | <0.001 | | | | | 0.001 | | | | | 0.001 |
| Absent | 46 | (75.4%) | 15 | (24.6%) | | 46 | (75.4%) | 15 | (24.6%) | | 46 | (75.4%) | 15 | (24.6%) | |
| Present | 56 | (96.6%) | 2 | (3.4%) | | 56 | (96.6%) | 2 | (3.4%) | | 56 | (96.6%) | 2 | (3.4%) | |
| Lymph node | | | | | <0.001 | | | | | <0.001 | | | | | <0.001 |
| metastasis | | | | | | | | | | | | | | | |
| Absent | 33 | (67.3%) | 16 | (32.7%) | | 33 | (67.3%) | 16 | (32.7%) | | 33 | (67.3%) | 16 | (32.7%) | |
| Present | 70 | (98.6%) | 1 | (1.4%) | | 70 | (98.6%) | 1 | (1.4%) | | 70 | (98.6%) | 1 | (1.4%) | |
| pT stage | | | | | 0.978 | | | | | 0.747 | | | | | 0.978 |
| pT1 | 62 | (87.3%) | 9 | (12.7%) | | 63 | (88.7%) | 8 | (11.3%) | | 62 | (87.3%) | 9 | (12.7%) | |
| pT2 | 21 | (80.8%) | 5 | (19.2%) | | 20 | (76.9%) | 6 | (23.1%) | | 21 | (80.8%) | 5 | (19.2%) | |
| pT3 | 15 | (83.3%) | 3 | (16.7%) | | 15 | (83.3%) | 3 | (16.7%) | | 15 | (83.3%) | 3 | (16.7%) | |
| pT4 | 5 | (100%) | 0 | | | 5 | (100%) | 0 | | | 5 | (100%) | 0 | | |
| Distant | | | | | 0.042 | | | | | 0.042 | | | | | 0.042 |
| metastasis* | | | | | | | | | | | | | | | |
| Absent | 84 | (83.2%) | 17 | (16.8%) | | 84 | (83.2%) | 17 | (16.8%) | | 84 | (83.2%) | 17 | (16.8%) | |
| Present* | 19 | (100%) | 0 | | | 19 | (100%) | 0 | | | 19 | (100%) | 0 | | |
| BRAF^{V600E} mutation | | | | | <0.001 | | | | | <0.001 | | | | | <0.001 |
| Negative | 22 | (56.4%) | 17 | (43.6%) | | 23 | (59.0%) | 16 | (41.0%) | | 22 | (56.4%) | 17 | (43.6%) | |
| Positive | 81 | (100%) | 0 | | | 80 | (98.8%) | 1 | (1.2%) | | 81 | (100%) | 0 | | |
| Recurrent or | | | | | 0.022 | | | | | 0.022 | | | | | 0.022 |
| persistent | | | | | | | | | | | | | | | |
| disease | | | | | | | | | | | | | | | |
| Absent | 79 | (82.3%) | 17 | (17.7%) | | 79 | (82.3%) | 17 | (17.7%) | | 79 | (82.3%) | 17 | (17.7%) | |
| Present | 24 | (100%) | 0 | | | 24 | (100%) | 0 | | | 24 | (100%) | 0 | | |
| ATA recurrence | | | | | <0.001 | | | | | <0.001 | | | | | <0.001 |
| risk | | | | | | | | | | | | | | | |
| Low | 16 | (50.0%) | 16 | (50.0%) | | 16 | (50.0%) | 16 | (50.0%) | | 16 | (50.0%) | 16 | (50.0%) | |
| Intermediate | 65 | (98.5%) | 1 | (1.5%) | | 65 | (98.5%) | 1 | (1.5%) | | 65 | (98.5%) | 1 | (1.5%) | |
| High | 22 | (100%) | 0 | | | 22 | (100%) | 0 | | | 22 | (100%) | 0 | | |
| AJCC stage | | | | | 0.099 | | | | | 0.099 | | | | | 0.099 |
| I | 78 | (83.0%) | 16 | (17.0%) | | 78 | (83.0%) | 16 | (17.0%) | | 78 | (83.0%) | 16 | (17.0%) | |
| II | 16 | (94.1%) | 1 | (5.9%) | | 16 | (94.1%) | 1 | (5.9%) | | 16 | (94.1%) | 1 | (5.9%) | |

| III | 0 | | 0 | 0 | | 0 | 0 | | 0 |
|---|---|---|---|---|---|---|---|---|---|
| IV | 9 | (100%) | 0 | 9 | (100%) | 0 | 9 | (100%) | 0 |

*Includes 17 synchronous and 2 metachronous metastases.
AJCC, American Joint Committee on Cancer; ATA, American Thyroid Association.

### INDUSTRIAL APPLICABILITY

The present disclosure relates to a method for detecting a thyroid cancer-specific DNA methylation biomarker and a composition thereof in order to provide information necessary for thyroid cancer diagnosis.

1. A method of analyzing a methylation level of a thyroid cancer biomarker CpG site of genomic DNA obtained from a sample to be analyzed in order to provide information necessary for diagnosis of thyroid cancer or determination of prognosis of thyroid cancer, the thyroid cancer biomarker CpG site being located in a gene selected from the group consisting of: (i) MICAL2 (Microtubule associated monooxygenase, calponin and LIM domain containing 2); (ii) LURAP1L-AS1 (LURAP1L antisense RNA 1); (iii) PKM2 (Pyruvate kinase M2); (iv) LTBP1 (Latent-transforming growth factor beta-binding protein 1); (v) MMP7 (Matrix metalloproteinase-7); (vi) Eukaryotic translation initiation factor 4E (EIF4E); (vii) DIAPH1 (Protein diaphanous homolog 1); and (viii) LOC100507487 (long intergenic non-protein coding RNA 2615).

2. The method of claim 1, wherein the thyroid cancer biomarker CpG site is at least one CpG site selected from the group consisting of cg10705422, cg15441605, cg24327132, cg16336556, cg17707274, cg00567113, cg06034194, cg21341586, cg26849pC382, and cg05763918, respectively represented by Illumina ID in HumanEPIC BeadChip.

3. The method of claim 1, wherein when the methylation level of the thyroid cancer biomarker CpG site is hypomethylated, the hypomethylated state indicates thyroid cancer or a poor prognosis of thyroid cancer.

4. The method of claim 1, wherein the diagnosis of thyroid cancer includes determining a stage of thyroid cancer, and the determination of the prognosis of thyroid cancer includes determining a recurrence rate of thyroid cancer.

5. The method of claim 1, wherein the methylation level analysis comprises a step of treating a genomic DNA obtained from a sample to be analyzed with bisulfate.

6. The method of claim 1, wherein the methylation level analysis comprises a step of amplifying a fragment comprising the thyroid cancer biomarker CpG site.

7. The method of claim 1, wherein the methylation level analysis comprises a pyrosequencing step.

8. A composition comprising a substance capable of analyzing a methylation level of a thyroid cancer biomarker CpG site located in a gene selected from the group consisting of: (i) MICAL2 (Microtubule associated monooxygenase, calponin and LIM domain containing 2); (ii) LURAP1L-AS1 (LURAP1L antisense RNA 1); (iii) PKM2 (Pyruvate kinase M2); (iv) LTBP1 (Latent-transforming growth factor beta-binding protein 1); (v) Matrix metalloproteinase-7 (MMP7); (vi) Eukaryotic translation initiation factor 4E (EIF4E); (vii) DIAPH1 (Protein diaphanous homolog 1); and (viii) LOC100507487 (long intergenic non-protein coding RNA 2615).

9. The composition of claim 8, wherein the thyroid cancer biomarker CpG site is at least one CpG site selected from the group consisting of cg10705422, cg15441605, cg24327132, cg16336556, cg17707274, cg00567113, cg06034194, cg21341586, cg26849382, and cg05763918, respectively represented by Illumina ID in HumanEPIC BeadChip.

10. The composition of claim 8, wherein the substance capable of analyzing the methylation level of the thyroid cancer biomarker CpG site is a primer pair capable of amplifying a fragment comprising the CpG site.

11. The composition of claim 10, further comprising a sequencing primer for sequencing an amplification product amplified by the primer pair.
